(19) United States
(12) Patent Application Publication
(10) Pub. No.: US 2021/0121314 A1
(43) Pub. Date: Apr. 29, 2021

(54) VENTRAL HERNIA BINDER WITH INSERT APPARATUS

(71) Applicant: Donald James BOYD, MATTAWA (CA)

(72) Inventor: Donald James BOYD, MATTAWA (CA)

(21) Appl. No.: 17/079,490

(22) Filed: Oct. 25, 2020

Related U.S. Application Data
(60) Provisional application No. 62/925,974, filed on Oct. 25, 2019.

(51) Int. Cl. A61F 5/03; A61F 2/00; A61F 5/24; A61F 5/34; A61N 1/04

(52) U.S. Cl. CPC A61F 5/03; A61F 2/0063; A61N 1/0484; A61F 5/34; A61N 1/0456; A61F 5/24

(57) ABSTRACT

The hernia binder of the present invention may include an insert that is placed between a hernia and a binder in order to prevent bulging of the hernia through a weakness in the abdominal wall muscles. The insert may be secured through various fasteners, including a hook and mesh fastener. The insert may be constructed in different sizes and shapes to conform to the patient's size, the specific hernia and other factors. The insert may also be constructed from various materials to accommodate these factors. The insert may be created from a mold of the specific patient who will utilize the present invention. The insert is therefore custom made and is constructed in different sizes and shapes to conform to the patient's size, the specific hernia and other factors.

### CROSS-REFERENCE TO RELATED APPLICATIONS

This application claims the benefit of U.S. patent application 62/925,974 FILED Oct. 25, 2019.

### FIELD OF THE INVENTION

The present invention relates generally to a hernia binder and more specifically relates to a ventral hernia binder and more specifically to a ventral hernia binder with insert.

### BACKGROUND

A ventral hernia is a hernia that forms in the abdominal wall, along the midline, due to a weakness in the muscles that creates a gap. Typically, with a ventral hernia, abdominal tissues or a portion of the intestines protrude into the gap. Many cases of ventral hernias occur at the site of a previous surgical incision. This is because the incision weakens the abdominal muscles, putting them at an increased risk of injury due to obesity, strain, and/or aging. A ventral hernia can occur immediately following surgery or even years after the procedure.

The type of ventral hernia diagnosed is defined based on where the hernia forms: incisional hernia which is a ventral hernia that develops at a previous incision site; epigastric hernia which is a ventral hernia that develops within the abdominal area between the bottom of the breast bone and the belly button; and an umbilical hernia which is a ventral hernia that develops in the area surrounding the navel or belly button.

As ventral hernias do not heal on their own, the treatment for a ventral hernia involves surgical intervention. The goal of ventral hernia surgery is to repair the gap in the abdominal wall and return the protruding tissues and organs to their natural position. The type of procedure performed will be determined by the physician after considering the severity of the hernia. There are two procedures that can be used to treat ventral hernias. Open ventral hernia repair which is an open hernia repair and is typically used for larger, more complex hernias. This surgery is performed by making one long incision near the hernia. In some cases, mesh made of animal tissue or a synthetic material may be used to hold the protruding tissues in place. Laparoscopic ventral hernia repair involves using a laparoscope, or a thin tube with a camera on the end, to guide the surgeon. During laparoscopic surgery, a surgeon will make three to four small incisions near the hernia. In some cases, mesh made of animal tissue or a synthetic material may be used to hold the protruding tissues in place. Laparoscopic repair is a minimally invasive technique that involves smaller incisions, a shorter time in the operating room, and faster recovery.

Recovery will vary for each patient, depending on the type of surgery performed and the overall health of the patient. Some patients may experience a low level of pain after an open repair procedure, while others may experience a high level of pain and discomfort, even after a minimally invasive laparoscopic procedure.

As part of the treatment and management of a ventral hernia, a belt or binder may be worn by the person. The current belts however are primarily made as a one size for all type of device and does not account for the varying types and sizes of hernias. Accordingly, a device is needed to address the shortcomings and disadvantages of the prior art.

CN200954163 discloses an air-inflated pressuring hernia treating belt. The utility model discloses an air inflated pressurized hernia therapy belt for hernia auxiliary therapy medical. The model comprises a hernia therapy belt body with a retaining strap and a connecting strap positioned on the lower side and two sides, three medicine pocket nylon retainers are arranged in order on the inner side of the hernia therapy belt body. Three air bags communicate with the air inflation device are positioned on the place where is corresponding to the medicine pocket nylon retainer on the hernia therapy belt. The hernia therapy belt can be used for therapy on the direct hernia, tilted hernia, abdominal hernia, outer abdominal hernia and an umbilical hernia. Air inflation on the air bag adjusts the medicine pocket pressure at the hernia position, and has the advantages of balancing the hernia position to absorb effectively Chinese herbs and body comfort.

CN204072490 discloses an inflatable medical abdominal binder. The utility model relates to solving the problem of poor treatment effect of existing medical abdominal binders. The inflatable medical abdominal binder comprises a strip-shaped binder body, a plurality of internal air bags, a tube capsule air valve and a handheld inflation ball, wherein the two end heads of the binder body are provided with corresponding nylon hook-loop fasteners; a medicine placement bag is arranged on the inner side at a central position of the binder body. During use, the handheld inflation ball is used for inflating the plurality of internal air bags closely attached to the abdomen of a human body with pressure; the air bags are closely attached to the abdomen of the human body after being expanded; the abdomen is squeezed to be maintained in a compressed state once a person performs abdomen in slightly, the pressure is adjustable and the power exists continuously so as to facilitate postpartum uterine contraction, cut fixation after cesarean section, slimming, waist support and shape adjustment.

US20050182344 discloses a recovery belt apparatus. The device includes a pad portion attached to a first belt portion and a second belt portion. The device has a removable attaching means for attaching the first belt portion with the second belt portion. The pad portion is adapted to be placed over either a person's abdomen or thoracic area, and the removable attaching means adjusts pressure of the pad portion on either a person's abdomen or thoracic area. Recovery belt apparatus includes pad portion coupled to a first belt portion on a first end and a second belt portion on a second end. Pad portion is of such a size to fit a surgical area of a person typical of surgical procedures, such as Cesarean section (C section), laparoscopic hiatal hernia, hernia, tummy tuck, hysterectomy, appendectomy, gall bladder surgery, thoracic surgery including lung or heart surgery.

CN205698235 discloses a pressurization binder for general surgical use. The utility model presses the belly and with the elastic strap. A cartridge bag can be filled with the traditional Chinese medicine, and an intermediate layer is filled with the gasbag. The air inlet on the gasbag, is provided with an end cap on the air inlet, the air inlet pressing the belly. The utility model discloses the beneficial effect through the control of the amount of aeration in the gasbag to adjust the elasticity between binder and the patient. The cartridge bag closely covers in the wound and Chinese herbal medicinal ingredient can promote wound healing in the cartridge bag to diminishing inflammation, shortening the patient's recovery time. The elastic strap is narrow in shape in width, and patient wears it thereby laminating the belly, prevent gliding by the elastic strap.

CN2082158 discloses a treatment binder for umbilical hernia. The utility model provides a baseband made by two pieces of pure cotton cloth that are folded and sewn, four elastic belts are sewn on the pure cotton cloth, bars with nylon hasps are sewn on the both ends of the baseband, sewing at the opposite side of a base strip, and a navel pad is also arranged at one end of the base strip. While the treatment binder for umbilical hernia is in use, the navel is pressed on the navel chakra of an infant, which is a wrapped circle for the treatment of umbilical hernia, and locked and combined by the bars with nylon hasps.

U.S. Pat. No. 9,220,585 discloses a hernia patch. The hernia patch supporting tissue in-growth conforms to a tissue wall upon surgical installation and fixation within a patient. The hernia patch can include a base and positioning straps. The base is formed of two layers that are affixed to each other around the perimeter of the patch, for example by stitching. A stabilizing washer is provided between the two layers, and the stitch is provided peripherally around the stabilizing washer, keeping the washer free-floating between the layers. The base, positioning straps, and stabilizing washer are formed of a structure that does not separate the layers of the implant or form a space in the form of a pocket, and promotes more uniform and confluent tissue incorporation or in-growth after implantation. The hernia patch may further include a hydrolysable bio-absorbable cross-linked coating of a fatty acid-based material, such as an omega-3 fatty acid based material.

US20070137642 discloses an infant stomach band to protect from injury. This band has particular usefulness in helping to prevent hernias in infants and preventing protruding belly buttons in infants. The band can have a especially useful applications for babies having colic. The cloth material is preferably stretchable and breathable while being a soft and comfortable material. Additional padding can be included beneath the cloth material and can be preferably positioned between a cloth material and elastic material to provide comfort to the infant. In a preferred embodiment the band compromises disposable materials substantially similar to the diaper materials used for infants' diapers. The band can include absorbent qualities as present in those in diapers while maintaining the predetermined geometrical shape to help prevent the hernia injuries and bulging belly button areas in infants.

U.S. Pat. No. 2,062,143 discloses an umbilical truss. The principal object of the invention is to provide an improved, more efficient and comfortable truss for the relief of conditions arising from umbilical hernia. A further object of the invention is to provide a truss of the type mentioned in which a balance is achieved between the bearing portion of the pad and the rear portion of the belt, thus increasing the efficiency, as well as the comfort of the truss in use. A further object of the invention is to provide in an umbilical truss a pad having a bearing head which is adapted to rotate upon a supporting stem, the head thus being adapted to remain constantly at the point of rupture during all conditions of movement by the user.

WO2018144456 discloses a hernia belt. A hernia belt may include a digitally knitted support belt and a two-part fastening system. The two-part fastening system may include a first part and a second part, in which the second part includes a plurality of fastener strips arranged spaced apart from each other. The digitally knitted support belt may include mesh stitch layouts for providing breathability and support.

U.S. Pat. No. 5,195,948 discloses an adjustable back support device. A back support device is disclosed comprising a belt structure designed to fit substantially around the waist of a user. An inflatable air bladder is attached inside the belt structure so that it is positioned adjacent to the lower back when the back support device is worn. Several embodiments of elastic strap members are disclosed which are attached between the air bladder and belt structure. The strap members enable the user to adjust the direction of force exerted by the air bladder on the lower back and helps to pull the air bladder around the waist. A manual air plump with two-way valve is positioned inside the belt structure near one end which enables the user to selectively adjust the amount of air in the air bladder while the back support device is worn. Using the above device, a method of providing back support is also disclosed.

All cited documents are incorporated by reference.

None of the above cited documents, alone or in combination satisfy the need for a hernia binder that has multipurpose insert pockets which efficiently and effectively provides abdominal and lumbar support and while not permitting bulging of the ventral hernia.

### BRIEF SUMMARY

It is an object of the invention to provide a ventral hernia binder with insert apparatus.

In accordance with an aspect of the invention there is provided a ventral hernia binder apparatus, comprising: a binder belt having an inner and outer surface; one or more means of fastening said binder on position on a patient; and a pouch positioned on said inner surface of said binder belt, said pouch being sized and positioned to correspond to a location of said ventral hernia, said pouch further being sized to accommodate an insert.

In accordance with another aspect of the invention there is provided a method of preventing the occurrence of a ventral hernia in a patient in need of such therapy using a ventral hernia binder apparatus having one more fasteners and one or more internal pouches, said method comprising the steps of: positioning said ventral hernia binder apparatus at an appropriate position to cover said ventral hernia; placing an insert in one or more of said one or more internal pouches; and securing said one more fasteners to hold said ventral hernia binder apparatus at said appropriate position on said patient.

In accordance with yet another aspect of the invention there is provided a method of preventing the recurrence of a ventral hernia in a patient in need of such therapy using a ventral hernia binder apparatus having one more fasteners and one or more internal pouches, said method comprising the steps of: positioning said ventral hernia binder apparatus at an appropriate position to cover said ventral hernia; placing an insert in one or more of said one or more internal pouches; and securing said one more fasteners to hold said ventral hernia binder apparatus at said appropriate position on said patient.

The advantages and features of the present invention will become better understood with reference to the following more detailed description and claims taken in conjunction with the accompanying drawings in which like elements are identified with like symbols.

To easily identify the discussion of any particular element or act, the most significant digit or digits in a reference number refer to the figure number in which that element is first introduced.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates the formation and subsequent repair of a ventral hernia.

FIGS. 2a and 2b illustrates placement of implanted mesh after a ventral hernia repair and subsequent 3D imaging of repaired hernia site.

FIGS. 3a and 3b illustrates views of aspects of embodiments of the subject matter in accordance with one embodiment of the invention.

FIG. 4 illustrates positioning of an abdominal binder in accordance with one embodiment of the invention.

FIGS. 5a and 5b illustrates front and back views of an abdominal binder, with insert pouch, in accordance with one embodiment of the invention.

FIG. 6 illustrates a diagrammatic front view of an embodiment of the present invention.

FIG. 7 illustrates a perspective front view of an additional embodiment of the present invention in use.

FIG. 8 illustrates an aspect of the subject matter in accordance with an alternative embodiment of the invention.

FIG. 9 illustrates an aspect of the subject matter in accordance with an alternative embodiment of the invention.

FIG. 10 illustrates a rear view of an additional embodiment of the present invention including lumbar support provided by an air bag.

FIG. 11 illustrates a rear view of an additional embodiment of the present invention including TENS technology.

FIG. 12 illustrates a rear view of yet an additional embodiment of the invention including support bands.

### DETAILED DESCRIPTION

The various embodiments of the present invention will hereinafter be described in conjunction with the appended drawings, wherein like designations denote like elements.

Devices and methods for carrying out the invention are presented in terms of embodiments depicted within the FIGS. However, the invention is not limited to the described embodiments, and a person skilled in the art will appreciate that many other embodiments of the invention are possible without deviating from the basic concept of the invention, and that any such work around will also fall under scope of this invention. It is envisioned that other styles and configurations of the present invention can be easily incorporated into the teachings of the present invention, and the configurations shall be shown and described for purposes of clarity and disclosure and not by way of limitation of scope.

A hernia is the bulging of the intestine or fat that pushes its way through a weakened area in the abdominal wall. There are more than one million hernia surgeries a year in the US, they may be located in the groin area, and are called inguinal hernias, or in the abdominal area, and are called ventral hernias. Men and women, even children can be affected, but men are the majority of hernia patients.

In view of the disadvantages inherent in the known art, the present invention provides a novel hernia binder. The present invention is superior to other hernia binders in that it efficiently and effectively provides abdominal and lumbar support while not permitting bulging of the ventral hernia. The abdominal prosthetic, as described herein, can be worn to prevent hernias from developing, or can be worn after surgery, with a custom made insert, to protect the herniated surgical site.

Hernias are typically repaired by either laparoscopic or open surgery. With laparoscopic repair, a small video camera is inserted through incisions in the abdominal wall. The cavity is inflated with carbon dioxide and the surgeon can pull the hernia back into place securing it with mesh and staples or tacks and glue. For open surgery, an incision is made in the abdomen and the hernia is pushed back into place and the abdominal muscle wall is stitched together, if the muscle is healthy, or by sewing in mesh patches if the area is weak.

In the field of major abdominal wall reconstruction, recurrence is the most important complication. In order to reduce recurrence rates, meshes are used as reinforcement of augmentation or bridging of large abdominal wall defects. Numerous meshes, including metal, polyester and biodegradable fabrics are available worldwide, differing in material, pore size, weight, tensile strength, elasticity and biocompatibility.

To help prevent hernia distention post-op and rupturing of the implanted mesh, a binder is often worn post-surgery by patients. However, known hernia binder belts often do not prevent the internal organs from pushing against the mesh and causing a bulging or distension when performing everyday activities such as picking up a heavy object or bending down. The hernia belt as described herein features a custom insert that provided additional support to the herniated site pre and post-op. By increasing the internal abdominal pressure and support in the region of weakened abdominal tissue, the various embodiments of the invention assist patients with preoperative issues, post op-physiotherapy and everyday activities.

The hernia binder of the present invention may include an insert that is placed between a hernia and a binder in order to prevent bulging of the hernia through a weakness in the abdominal wall muscles. The insert may be constructed in different sizes and shapes to conform to the patient's size, the specific hernia and other factors. The insert may also be constructed from various materials to accommodate these factors. The insert may be created from a mold of the specific patient who will utilize the present invention. The insert is therefore custom made and is constructed in different sizes and shapes to conform to the patient's size, the specific hernia and other factors. The insert may be secured to the binders through various fasteners, including a hook and mesh fastener, sometimes known as Velcro.

The hernia binder may also include an airbag or bladder to provide additional support to the patient, including lumbar support and abdominal wall support. In some embodiments, the hernia binder may also include airbag or bladder to provide additional support to the patient, including lumbar support and abdominal wall support. An air bag nozzle may be located on the front side of the apparatus to allow for easy patient access to control the amount of air in the bladder.

In some embodiments of the invention, the binder of the present invention may include one or more flexible, but resistant, bands or rods that are embedded within the binder and/or insert or insert pouch. These bands will provide additional support to a patient in order to keep the patient in a more vertical position in order to assist with the daily movement of the patient.

The support bands or rods are multifunctional and can be inserted in many different positions in the binder. The support rods or bands may be used in the region of the tissue that is at risk of becoming herniated or, when a hernia has been repaired, they may be positioned to support and prevent recurrence. The support rods or bands may be used in the lumbar region to support the musculature in the lower back and relieve stress and pain.

In some embodiments of the invention, the support rods or bands that are installed in the anterior of the binder cooperate with the support rods or bands that are installed in the posterior of binder. In this embodiment, the anterior rods or bands protect and support the ventral musculature when the patient is performing everyday tasks, such as bending forward. In this embodiment, the posterior rods or bands similarly protect and support the ventral musculature, and function in an opposing and off-setting manner to the anterior rods or bands, to assist in returning the body to an upright position.

In alternative embodiments of the hernia binder, the apparatus may take the form of a vest or a full upper body garment and may include groin of thigh support elements. Support rods may be incorporated into pouches or sleeves in various positions within the binder, for example, some patients having neck and upper spinal lateral cord issues in the C5-C7 region may benefit from additional support to the pectoralis major and minor muscles, via the lateral and medial pectoral nerves, as well as the coracobrachialis, brachialis and biceps brachii, via the musculocutaneous nerve.

The support rods or bands may be fabricated from various metals or plastics, typically including steel, stainless steel, copper or any medical grade plastic having the appropriate strength. The rods or bands may be coated in a protective material such as foam, rubber or fabric. In instances, where the hernia repair has resulted in removal of large quantities of musculature, the custom-made insert may contain one of more support rods or bands to hold the insert in shape and provide additional support to the weakened musculature in the herniated region.

The features of the invention which are believed to be novel are particularly pointed out in the specification. The present invention now will be described more fully hereinafter with reference to the accompanying drawings, which are intended to be read in conjunction with both this summary, the detailed description and any preferred and/or particular embodiments specifically discussed or otherwise disclosed. This invention may, however, be embodied in many different forms and should not be construed as limited to the embodiments set forth herein; rather, these embodiments are provided by way of illustration only and so that this disclosure will be thorough, complete and will fully convey the full scope of the invention to those skilled in the art.

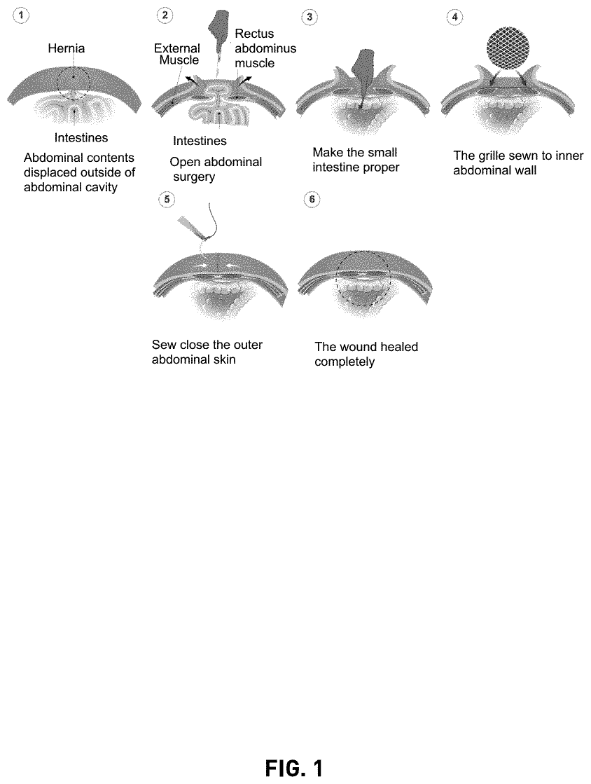

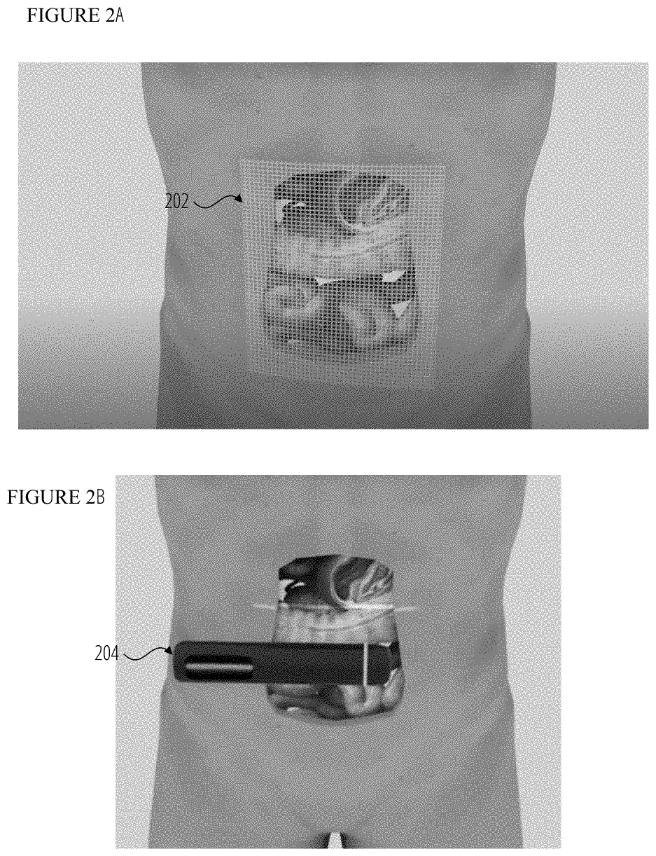

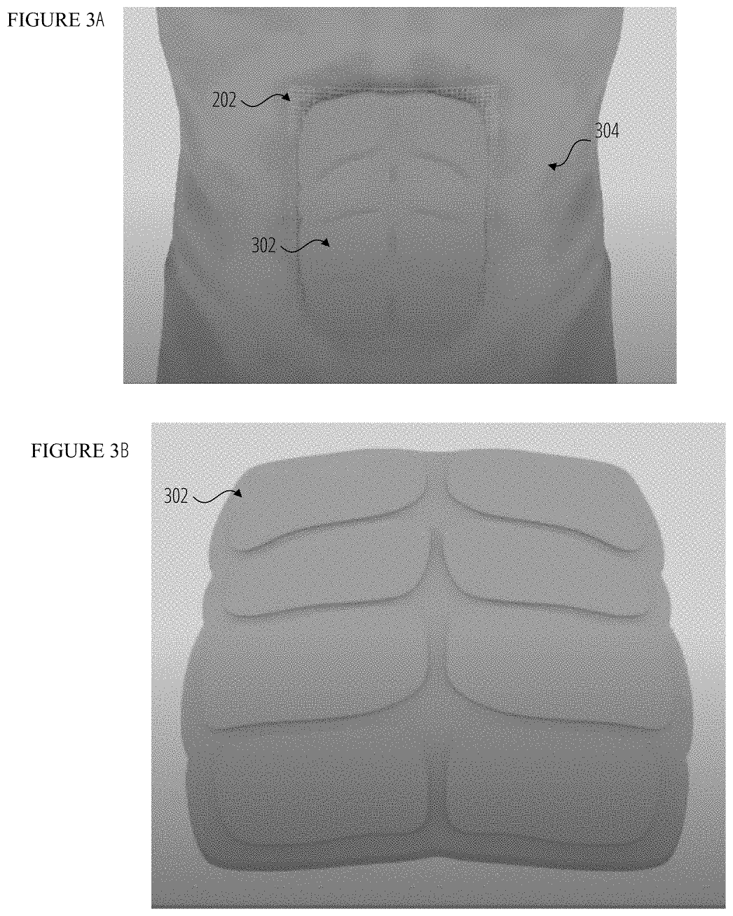

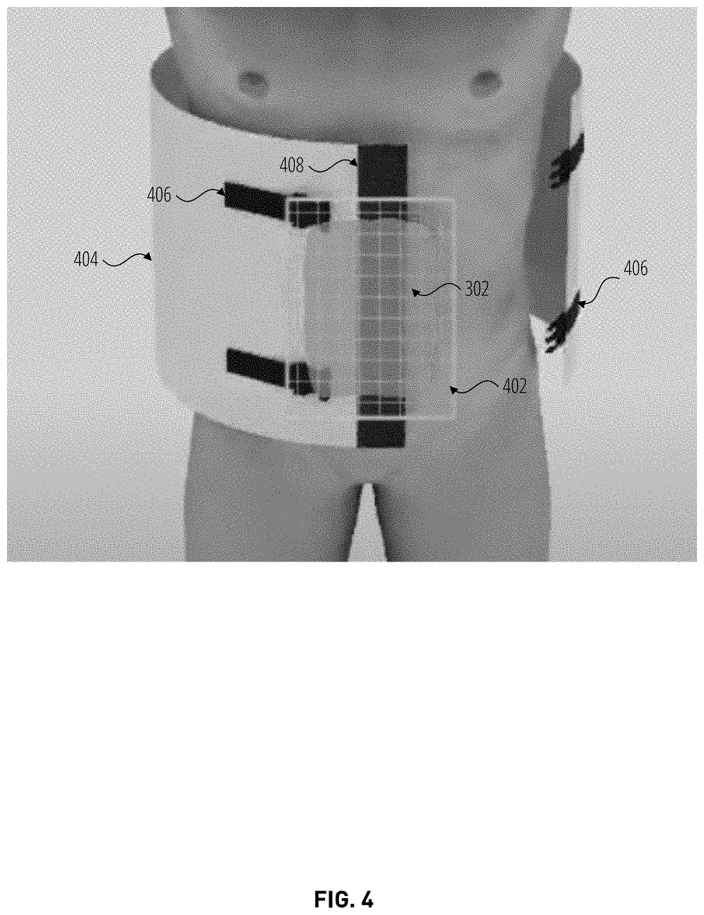

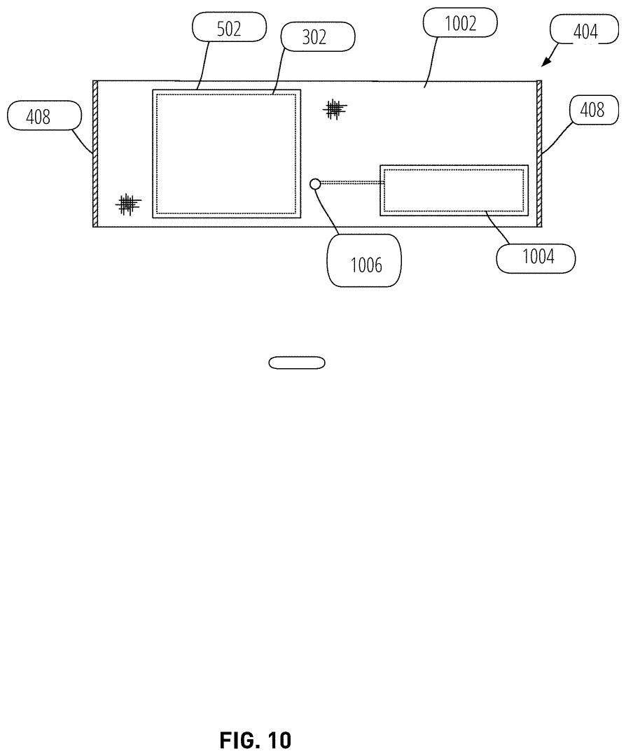

Referring now to the drawings, there is shown in FIGS. 1-2 the anatomy and correction of a ventral hernia. FIG. 3 shows a custom insert in accordance with an embodiment of the invention. FIGS. 4-9 show various different embodiments of the binder apparatus. FIGS. 10-12 show various different additional features of the ventral hernia binder of the present invention.

FIG. 1 illustrated the various stages of ventral hernia repair. FIG. 1.1 shows a hernia formed under the skin due to the intestines or abdominal contents being displaced outside the abdominal cavity. FIG. 1.2 illustrates the exposure of herniated tissue during an open abdominal surgery. FIG. 1.3 shows the herniated tissue, including fat and/or intestines, being re-inserted back into the abdominal cavity. FIG. 1.4 indicates the placement of a grille or mesh patch being implanted on the inner surface of the abdominal wall. FIG. 1.5 shows the surgical repair of the initial incision wound. FIG. 1.6 shows a healed hernia.

FIG. 2A illustrates a pictorial view of the abdomen and internal organs. After a hernia has been repaired, a layer of implanted mesh 202 is secured in place on the inner surface of the abdominal wall (not shown).

FIG. 2B illustrates a method of producing a 3D image, in this embodiment by use of a 3D measuring device 204 such as a 3D-laser. Other techniques known in the medical filed would include, but are not limited to, plaster casting, ultrasound, MRI's or CAT scans.

FIG. 3A shows the positioning of a custom insert 302 that has been produced in accordance with one of the known techniques outlined above. The custom insert 302 is positioned over the approximate area of the weakened area of the abdomen 304 that has the implanted mesh 202 attached beneath it.

FIG. 3B shows a close-up representation of a custom insert 302. The custom inserts 302 can be fabricated from flexible or more rigid material, and they are washable.

FIG. 4 shows a diagrammatic representation of mesh 402 and it relative positioning within the abdominal cavity. The position of the custom insert 302, over the implanted mesh 202 can be seen. A binder 404 of an embodiment of the invention can be seen being sized and fitted to the patient. The binder 404 is secured in place by way of first securement means 408 and first securement means 408.

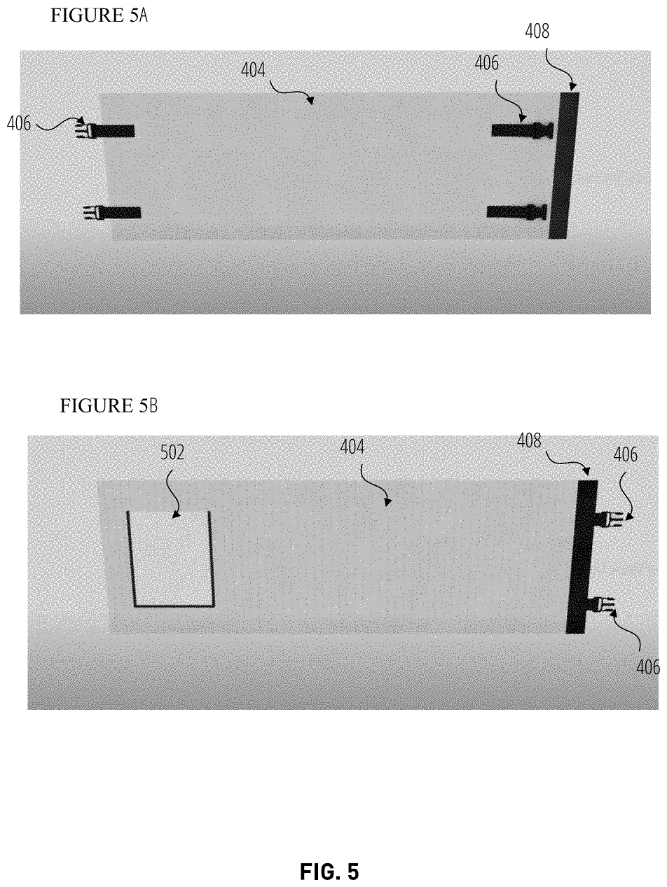

FIG. 5A shows a front or outer side view of a binder 404 in accordance with an embodiment of the invention. The first securement means 408 and second securement means 406 can be seen.

FIG. 5B shows a rear or inner side view of a binder 404 in accordance with an embodiment of the invention. The custom positioned insert pouch 502 can be seen to correspond with the position of the implanted mesh 202 (not shown).

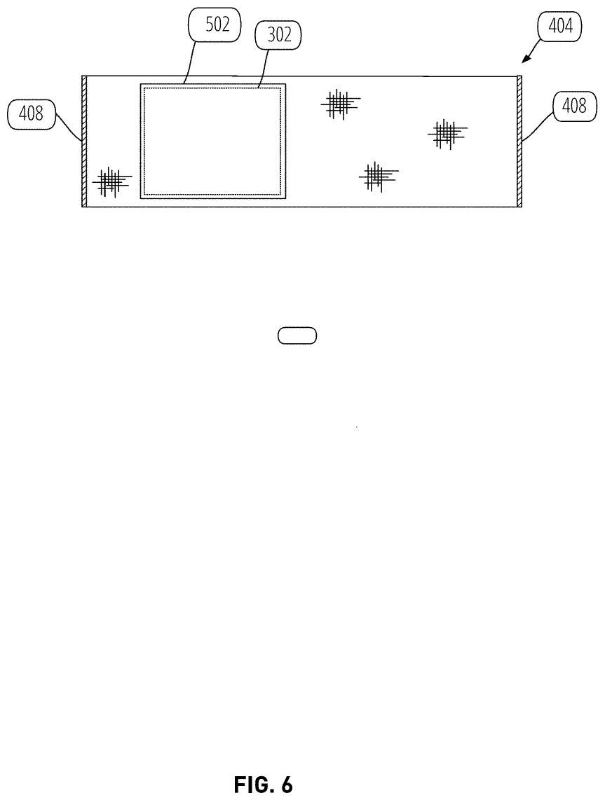

FIG. 6 shows a rear or inner side view of an embodiment of the binder 404. In this embodiment, the first securement means 408 are fabricated from a strip of Velcro™. Again, the insert pouch 502 containing the custom insert 302 can be seen.

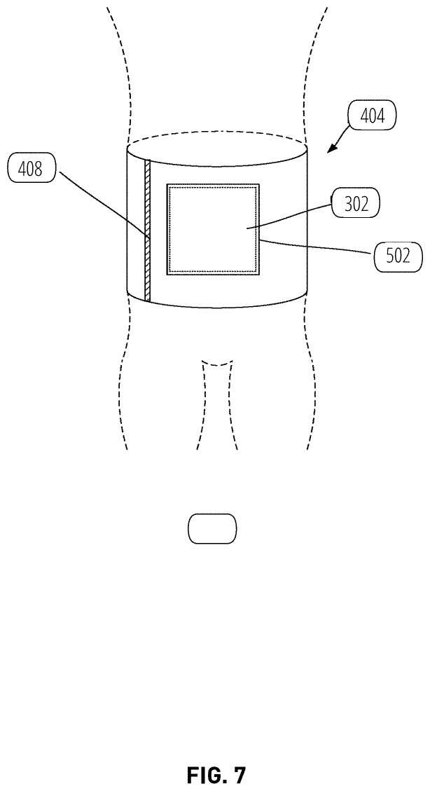

FIG. 7 shows the relative positioning of a binder 404 of an embodiment of the invention when worn in the abdominal region of the body. The inner positioning of the insert pouch 502 and custom insert 302 are indicated.

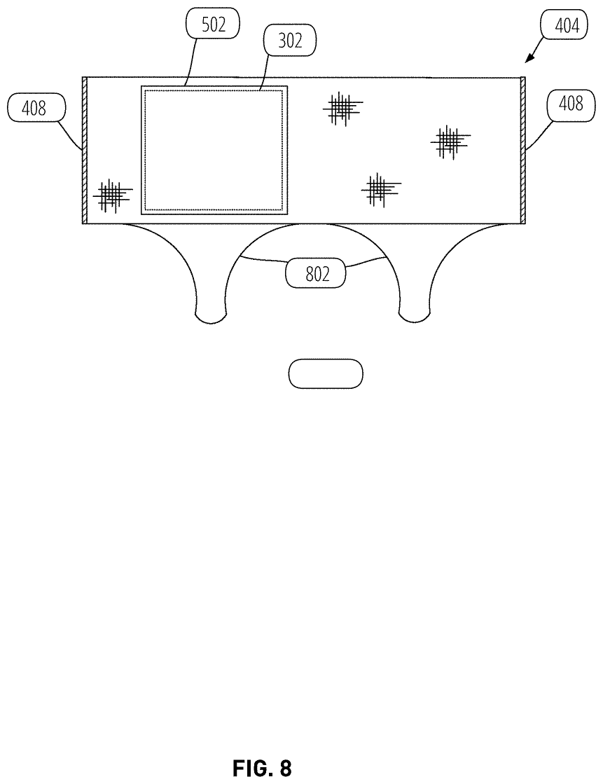

FIG. 8 illustrates an alternative embodiment of a binder of the present invention. The binder 404 comprises a first securement means 408, and optional first securement means 408 (not shown) and the inner insert pouch 502 and custom insert 302. However, this embodiment of the binder additionally has a groin support 802 member.

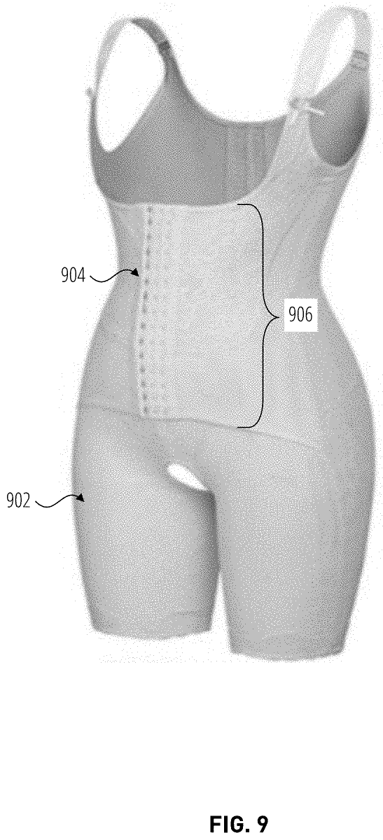

FIG. 9 illustrates full body compression outfit comprising an extended binder 906 which may be suitable for a multiple hernia patient. The extended binder 906 or full body suit is custom fitted to the patient and may comprise thigh members 902 extend around upper portions of the legs.

FIG. 10 shows an embodiment of a hernia binder 404 essentially as illustrated in FIG. 6, however the hernia binder 404 shown in this fig also features an air bag/bladder 1004 in communication with an air bag/bladder nozzle 1006. The positioning of the air bag/bladder 1004 is such that it will provide lumbar support. In alternative embodiments of the invention, the air bag/bladder 1004 can be positioned in the region of the herniated tissue in order to provide additional support to the custom insert 302.

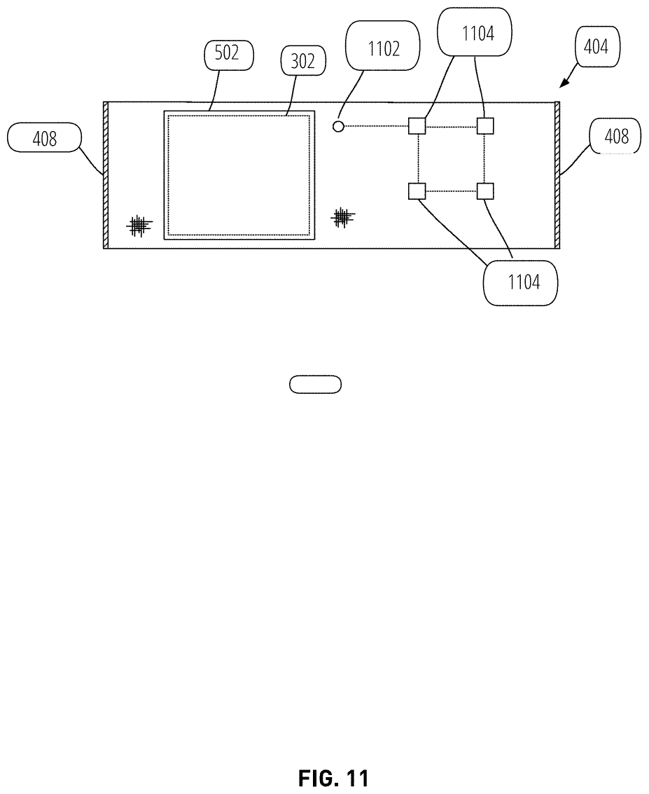

FIG. 11 shows an embodiment of a hernia binder 404 essentially as illustrated in FIG. 6, however the hernia binder 404 shown in this fig also features a transcutaneous electrical nerve stimulation (TENS) treatment device. The TENS device comprises one or more TENS unit pads 1104 and a TENS unit switch 1102 to activate the TENS device. These components comprise standard TENS electrical unit electrode pads, wiring, controller hardware, batteries and other standard TENS unit components. This embodiment of the invention may be configured to comprise a TENS unit pouch (not shown) to safely and securely retain the TENS device in an appropriate position within the binder 404.

Transcutaneous electrical nerve stimulation (TENS) therapy involves the use of low-voltage electric currents to treat pain. A small device delivers the current at or near nerves. TENS therapy blocks or changes perception of pain. A TENS unit consists of a battery-powered device that delivers electrical impulses through electrodes placed on the surface of the skin. The electrodes are placed at or near nerves where the pain is located or at trigger points.

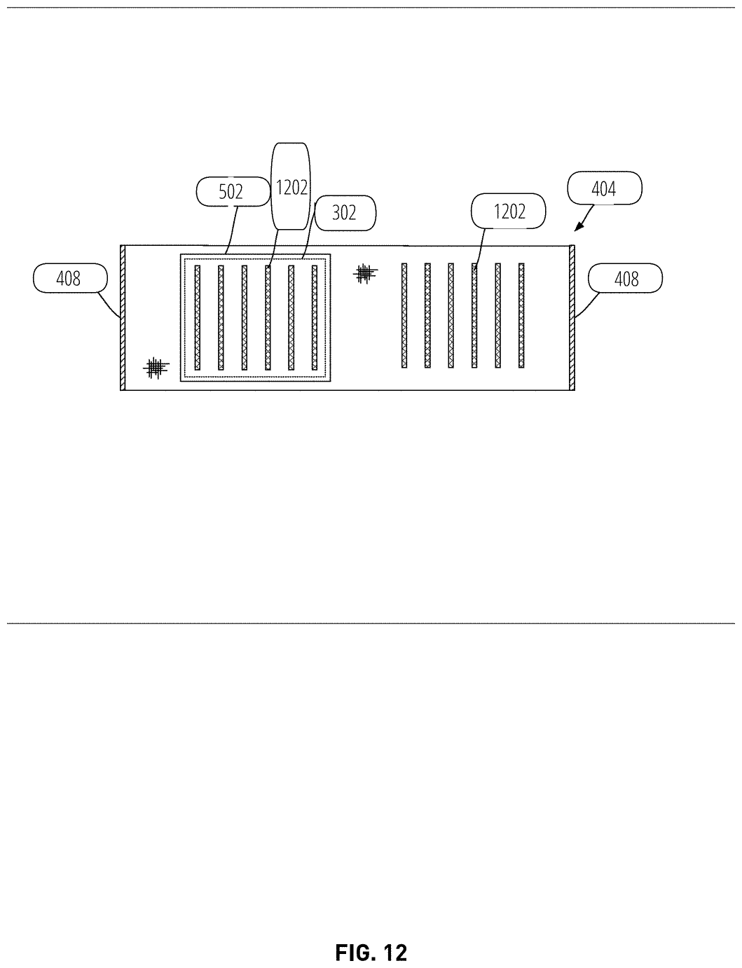

FIG. 12 illustrates shows an embodiment of a hernia binder 404 essentially as illustrated in FIG. 6, however the hernia binder 404 shown in this fig also features a number of support bands 1202. In the embodiment as illustrated, some of the support bands 1202 are situated in the region of the herniated tissue and some are located in the lumbar region of the back to provide additional support and relieve back pain.

As described in conjunction with the figures, the present invention is directed to a ventral hernia binder having an insert. More specifically, an abdominal hernia binder/belt, with insert to create gap between the surgery area or an area with weakened musculature and a binder/belt. The binder or belt may have an airbag or inflatable insert or pad insert in the region of the hernia. The binder or belt may have an airbag or inflatable insert, or support or structural bands in the lumbar area to provide back support. The binder or belt may also include one or more pouches or tabs to hold elements of a TENS device in place.

As shown in FIGS. 1-12, embodiments of a hernia binder apparatus of the present invention may comprise a binder that extends around the torso of a patient. The exact specifications, materials used, and method of use of the hernia binder may vary upon manufacturing. The binder material is to be constructed from a fabric that is strong and supportive, while providing flex and a minimal level of extensibility in line with other binders 404 known in the field.

The binder may be secured by one or more different types of fasteners. In one embodiment, a hook and mesh fastener may be used, otherwise known as Velcro™. In other embodiments, buckles and belts may also be employed. Clip fasteners, as shown in FIGS. 4 and 5 may secure the binder in place. Fasteners such as buckles, belts and clips may be used alone or in conjunction with known hook and loop fasteners.

In some embodiments, the binder and/or insert of the present invention may include components to allow for transcutaneous electrical nerve stimulation (TENS) treatment of the hernia site. These components may include TENS electrical unit electrode pads, wiring, controller hardware, batteries and other standard TENS unit components. The ability to provide TENS treatment can assist the patient in the healing of the hernia site.

The binder includes one or more pouches or pockets to holes the various additional features in place. The additional features may include a hernia custom insert 302, an air bag/bladder 1004 and TENS equipment. The pouches or pockets may also feature means of holding wires or tubes in place. Such means may be poppers, buttons, and hooks, and include loops or sleeves of fabric.

The custom insert 302 is to be made according to methods, and materials, known in the field, including molding and 3D printing methods. The area of weakens tissue, often corresponding to the area of implanted mesh 202, measured by techniques known in the field including but not limited to laser 3D imagery, plaster casting, or MRI to determine the exact shape and hernia depression over the mesh. Once the insert is placed inside the binder, it helps keep the mesh from distending while performing strenuous activities that wouldn't be done normally.

In accordance with additional embodiments of the invention, the binder may include several other features to support or ease pain in the lumbar region of the body. These may include a pump-up air bladder or somewhat rigid support bands to support the lumbar region. The air bladder or support bands are typically fitted inside a corresponding pocket or pouch in the binder 404.

In accordance with further embodiments of the invention, the binder may include an pump up air bladder or somewhat rigid support bands to support the weakened herniated tissue. The air bladder or support bands are typically fitted inside a corresponding pocket or pouch in the binder 404 to add additional support to the custom insert 302.

In accordance with yet additional embodiments of the invention, the binder may also be specifically adapted to support and retain various equipment associated with the installation of a TENS device. The TENS device being positioned in the lumbar region to relieve back pain.

In some embodiments, the apparatus may include various other support structures, such a groin support.

The binder is easy to wash, the insert may be flexible or solid and is also washable, can be made to fit different type and sizes of ventral hernias, as well as work with different binder models.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The exemplary embodiment(s) were chosen and described in order to best explain the principles of the present invention and its practical application, to thereby enable others skilled in the art to best utilize the present invention and various embodiments with various modifications as are suited to the particular use contemplated.

The foregoing descriptions of specific embodiments of the present invention have been presented for purposes of illustration and description. They are not intended to be exhaustive or to limit the invention and method of use to the precise forms disclosed. Obviously, many modifications and variations are possible in light of the above teaching. The embodiments described were chosen and described in order to best explain the principles of the invention and its practical application, and to thereby enable others skilled in the art to best utilize the invention and various embodiments with various modifications as are suited to the particular use contemplated. It is understood that various omissions or substitutions of equivalents are contemplated as circumstance may suggest or render expedient, but is intended to cover the application or implementation without departing from the spirit or scope of the claims of the present invention.

What is claimed is:

1. A ventral hernia binder apparatus, comprising:
a binder belt having an inner and outer surface;
one or more means of fastening said binder on position on a patient; and
a pouch positioned on said inner surface of said binder belt, said pouch being sized and positioned to correspond to a location of said ventral hernia, said pouch further being sized to accommodate an insert.

2. The ventral hernia binder apparatus of claim 1, further comprising a custom made insert that is shaped to correspond to the ventral hernia.

3. The ventral hernia binder apparatus of claim 2, wherein said insert additionally comprises support bars.

4. The ventral hernia binder apparatus of claim 2, wherein said support bars are fabricated from metal or plastic.

5. The ventral hernia binder apparatus of claim 1, wherein said one or more means of fastening are selected from the group consisting of; hook and loop, buckles, belts, clips and clasps.

6. The ventral hernia binder apparatus of claim 1, wherein said binder belt is washable.

7. The ventral hernia binder apparatus of claim 1, wherein said binder belt additionally comprises a groin support.

8. The ventral hernia binder apparatus of claim 1, wherein said binder belt additionally comprises an air bag or bladder apparatus.

9. The ventral hernia binder apparatus of claim 8, wherein said binder belt is configured to accommodate said air bag or bladder apparatus.

10. The ventral hernia binder apparatus of claim 8, wherein said air bag or bladder apparatus is positioned at the location of said ventral hernia or in a lumbar region of said patient.

11. The ventral hernia binder apparatus of claim 1, wherein said binder belt additionally comprises one or more support bands.

12. The ventral hernia binder apparatus of claim 11, wherein said binder belt is configured to accommodate said support bands.

13. The ventral hernia binder apparatus of claim 11, wherein said support bands are positioned at the location of said ventral hernia or in a lumbar region of said patient.

14. The ventral hernia binder apparatus of claim 1, wherein said binder belt is configured to accommodate a TENS unit.

15. The ventral hernia binder apparatus of claim 14, wherein said binder belt is configured to accommodate said TENS unit.

16. The ventral hernia binder apparatus of claim 14, wherein said TENS unit is positioned at the location of said ventral hernia or in a lumbar region of said patient.

17. A method of preventing the occurrence of a ventral hernia in a patient in need of such therapy using a ventral hernia binder apparatus having one more fasteners and one or more internal pouches, said method comprising the steps of:
positioning said ventral hernia binder apparatus at an appropriate position to cover said ventral hernia;
placing an insert in one or more of said one or more internal pouches; and
securing said one more fasteners to hold said ventral hernia binder apparatus at said appropriate position on said patient.

18. The method of claim 17, comprising the additional step of:
placing additional features in one or more of said one or more internal pouches, said additional features being selected from the group consisting of; an air bag or bladder, support bars, and a TENS unit; and
positioning said one or more internal pouches in appropriate positions on said patient, prior to said step of securing said one more fasteners.

19. A method of preventing the recurrence of a ventral hernia in a patient in need of such therapy using a ventral hernia binder apparatus having one more fasteners and one or more internal pouches, said method comprising the steps of:
positioning said ventral hernia binder apparatus at an appropriate position to cover said ventral hernia;
placing an insert in one or more of said one or more internal pouches; and
securing said one more fasteners to hold said ventral hernia binder apparatus at said appropriate position on said patient.

20. The method of claim 19, comprising the additional step of:
placing additional features in one or more of said one or more internal pouches, said additional features being selected from the group consisting of; an air bag or bladder, support bars, and a TENS unit; and
positioning said one or more internal pouches in appropriate positions on said patient, prior to said step of securing said one more fasteners.
